(19) United States
(12) Patent Application Publication
(10) Pub. No.: US 2003/0109036 A1
(43) Pub. Date: Jun. 12, 2003

(54) Method for differentiating islet precursor cells into beta cells

(75) Inventor: Frederick D. Wu, West Sacramento, CA (US)

Correspondence Address: Audrey A. Millemann, Weintraub Genshlea Chediak Sproul Law Corporation, 11th Floor, 400 Capitol Mall, Sacramento, CA, 95814, US

(73) Assignee: THE REGENTS OF THE UNIVERSITY OF CALIFORNIA, Oakland, CA

(21) Appl. No.: 10/251,004

(22) Filed: Sep. 19, 2002

Related U.S. Application Data
(60) Provisional application No. 60/340,992, filed on Dec. 6, 2001.

(51) Int. Cl. C12N 5/08

(52) U.S. Cl. 435/366

(57) ABSTRACT

The invention includes a method of differentiating pancreatic islet stem cells or islet precursor cells into functioning beta cells to treat diabetes mellitus by transplanting the cells into a diabetic animal. Pancreatic cells are isolated and cultured such that the population of nestin-positive cells increases. The cells are then cultured on poly-D-lysine such that cell aggregates form. The cell aggregates are transplanted into a diabetic animal, where they produce insulin and lower blood glucose concentrations.

### CROSS-REFERENCE TO RELATED APPLICATION

The present application claims the benefit of U.S. provisional patent application No. 60/340,992, filed Dec. 6, 2001 and entitled “Method for Differentiating Islet Precursor Cells into Beta Cells,” which is hereby incorporated by reference.

### BACKGROUND OF THE INVENTION

1. Field of the Invention

The invention relates to methods for differentiating pancreatic islet stem cells or islet precursor cells into beta cells and the treatment of diabetes mellitus by the transplantation of such cells.

2. Description of Related Art

Diabetes is a disease characterized by elevated blood glucose concentrations that, when left untreated, may lead to a myriad of medical problems including coma, cardiovascular disease, peripheral neuropathy, and blindness. Both type I and type II forms of the disease result from defects in insulin promoted tissue uptake of blood glucose. Type I diabetics no longer secrete insulin in response to hyperglycemia after autoimmune destruction of insulin producing beta cells in their pancreatic Islets of Langerhans. (The islets are clusters of multiple cell types, including functioning glucose-sensitive, insulin-secreting cells, also called beta cells.)

The transplantation of pooled islets from human cadavers has been shown to lead to normal blood glucose concentrations without exogenous insulin. The two major problems with transplantation have been the immune response of the recipient and the supply of donor islet cells. Recently, the combination of a non-steroidal anti-rejection drug regimen and increased islet mass used for transplantation into the liver has helped to minimize the problem of rejection. The supply of donor islet cells remains a key limiting factor.

Type I diabetes is an ideal target for stem cell based therapy because the disease results from the loss of a single cell type. It is generally accepted that islets are formed from precursor or stem cells in the pancreas. Both mouse and human adult pancreas derived cells have been propagated in culture and stimulated in vitro to differentiate into cells that exhibit characteristics similar to those of differentiated islet cells. (Bonner-Weir, S., M. Taneja, G. C. Weir, K. Tatarkiewicz, K. Song, A. Sharma, and J. J. O'Neil. In vitro cultivation of human islets from expanded ductal tissue. Proc. Nat. Acad. Sci. 9, no. 14 (2000): 7999-8004; Cornelius, J. G., V. Tchernev, K. J. Kao, and A. B. Peck. In vitro-generation of islets in long-term cultures of pluripotent stem cells from adult mouse pancreas. Horm Metab Res. 29, no. 6 (1997): 271-77; Ramiya, V. K., M. Maraist, K. E. Arfors, D. A. Schatz, A. B. Peck, and J. G. Cornelius. Reversal of insulin-dependent diabetes using islets generated in vitro from pancreatic stem cells. Nat Med. 6, no. 3 (2000): 278-82.) When transplanted into diabetic mice, adult mouse derived cells were able to maintain normoglycemia (Ramiya et al., “Reversal of insulin-dependent diabetes,” 278-82), suggesting that some of these transplanted cells terminally differentiated into insulin producing beta cells. Embryonic mouse stem cells have been cultured and caused to differentiate into islet-like cells, but, when transplanted into diabetic mice, did not control diabetes. (Lumelsky, N., O. Blondel, P. Laeng, I. Velasco, R. Ravin, and R. McKay. Differentiation of embryonic stem cells to insulin-secreting structures similar to pancreatic islets. Science 292, no. 5520 (2001): 1389-94.) Attempts to culture functional adult human islets in vitro, however, have failed to produce functional islets because the proliferation of the beta cells resulted in the loss of physiological function. (Beattie, G. M., J. S. Rubin, M. I. Mally, T. Otonkoski, and A. Hayek. Regulation of proliferation and differentiation of human fetal pancreatic islet cells by extracellular matrix, hepatocyte growth factor, and cell-cell contact. Diabetes 45 (September 1996): 1223-28; Beattie, G. M., V. Cirulli, A. D. Lopez, and A. Hayek. Exvivo expansion of human pancreatic endocrine cells. J Clin Endo and Met 82, no. 6 (1997): 1852-56.)

There is evidence that nestin is a marker for islet stem cells or islet precursor cells. In mice, both adult pancreas cells and embryonic stem cells, in differentiating into islet-like cells, progressed through an intermediate stage of cell differentiation in which the cells were nestin-positive. (Zulewski, H., E. J. Abraham, M. J. Gerlach, P. B. Daniel, W. Moritz, B. Muller, M. Vallejo, M. K. Thomas, and J. F. Habener. Multipotential nestin-positive stem cells isolated from adult pancreatic islets differentiate ex vivo into pancreatic endocrine, exocrine, and hepatic phenotypes. Diabetes 50 (March 2001): 521-33; Lumelsky et al., “Differentiation of embryonic stem cells,” 1389-94.) Nestin-positive cells have also been found in human pancreas. (Hunziker, E. and M. Stein. Nestin-expressing cells in the pancreatic islets of Langerhans. Biochem Biophys Res Commun 271 (April 2000): 116-19.)

Thus, there is a need for a method of generating islets in culture that can be transplanted into a diabetic to function to control the diabetes. In particular, there is a need for a method of easily propagating islet stem cells or islet precursor cells in culture and causing them to differentiate into insulin-producing beta cells.

### SUMMARY OF THE INVENTION

The present invention is directed to solving the problem of the limited supply of available islet cells. The method of the invention provides a way in which islet stem cells or islet precursor cells can be cultured such that they proliferate and are then stimulated to differentiate into islets that will function to control diabetes once transplanted.

The method of the invention comprises culturing pancreatic cells, culturing the cells to cause the population of nestin-positive cells to increase, culturing the nestin-positive cells on a substrate of poly-D-lysine such that the cells form cell aggregates, and transplanting the nestin-positive cell aggregates into a diabetic animal.

### BRIEF DESCRIPTION OF THE DRAWINGS

FIG. 1 shows the mean blood glucose concentrations over time of mice treated with one of three procedures: insulin pellets only, nestin-positive cell aggregates and insulin pellets concurrently, and insulin pellets followed by nestin-positive cell aggregates two days later.

FIG. 2 shows the blood glucose concentrations of the individual mice described in FIG. 1 which were treated with insulin pellets only.

FIG. 3 shows the blood glucose concentrations of the individual mice described in FIG. 1 which were treated with nestin-positive cell aggregates and insulin pellets concurrently.

FIG. 4 shows the blood glucose concentrations of the individual mice described in FIG. 1 which were treated with insulin pellets followed by nestin-positive cell aggregates two days later.

### DETAILED DESCRIPTION OF THE PREFERRED EMBODIMENT

Method for Differentiating Islet Precursor Cells into Beta Cell

The method of the invention includes the following steps. Cells obtained from the pancreas are cultured. The cells may be obtained from a fetus or adult animal; the animal may be a human, mouse, dog, cat, or other mammal.

The pancreatic cells are cultured such that, as they proliferate, the population of cells that express nestin increases. In other words, the culture is enriched for cells that express nestin, as indicated by nestin-positive staining. This is preferably accomplished by conscientious neglect or by sub-cloning, by methods known to those skilled in the art, or by other means. These nestin-positive cells do not express insulin.

The cells are then cultured on a substrate of poly-D-lysine. This results in the nestin-positive cells forming cell aggregates that express insulin. Thus, after culturing in poly-D-lysine, the nestin-positive cells acquire insulin expression, indicating that poly-D-lysine has a differentiating effect on the nestin-positive cells and that the nestin-positive cells are islet precursor cells or islet stem cells.

The aggregates of nestin-positive cells are then transplanted into a diabetic animal, such as a human, mouse, dog, cat, or other mammal, by methods known to those skilled in the art, including by implanting the cell aggregates beneath the kidney capsule, into the liver, or into other receptive organs. It is preferable to transplant the nestin-positive cell aggregates into an animal whose blood glucose concentration is well-controlled. Once the aggregates of nestin-positive cells are transplanted into the diabetic animal, they function as beta cells by secreting insulin to control blood glucose concentration.

Example of Method as Used in Human Cells

Because it is clear that, during fetal development, islet cells differentiate from precursor cells, fetal pancreatic tissue was used as an enriched source of islet precursor cells. Nestin-positive cells were isolated from human fetal pancreas. These cells were maintained in culture for over two years as epithelioid monolayers and remained undifferentiated for over 15 population doublings. The cells were then cultured on poly-D-lysine and stimulated to form islet-like cell aggregates with insulin expression. When transplanted into diabetic mice, these cell aggregates maintained glucose concentrations below 200 mg/dl.

Pancreatic Tissue

Primary cultures of human fetal pancreas were established through mechanical disruption and seeding of tissue onto tissue culture plates, resulting in monolayers of diverse cell types. Human fetal pancreas (HFP, 21 weeks gestation) was acquired from Advanced Bioscience Resources (Alameda, Calif.). After harvesting, tissue was immediately placed in cold (0°) RPMI 1640 medium. Within five hours, tissue was minced into 1 mm³ pieces and washed in RPMI.

Tissue Culture

Minced tissue was placed in T-25 tissue culture flasks with RPMI +5% fetal bovine serum (FBS), 100 mM penicillin, and 10 mM streptomycin. The medium was changed on the third and sixth day post isolation. Dissociated tissue and cells were left for four weeks without media change. After four weeks, the media was changed weekly.

Tissue was cultured in RPMI (Gibco) +10% FBS (Sigma), at 37° C. and 5% CO₂. Monolayers were dissociated in Ca²⁺/Mg⁺ free phosphate buffered saline (PBS) containing 0.05% trypsin and 0.02% EDTA (Gibco) for 10 minutes at 37° C. Trypsin was inactivated by addition of serum containing medium. Cells were split 1:4 every three weeks.

Isolation of Nestin-Positive Cells

Cell cultures established from primary pancreatic tissue were analyzed to determine the types of cells present. Cultured cells were fixed in cold (−20° C.) methanol for 10 minutes and allowed to air dry. Cell population analysis by histology was performed using antibodies to cytokeratin 19 (1:200 Santa Cruz Biotechnology), antibodies to vimentin (1:100 Santa Cruz), antibodies to insulin (1:100 Santa Cruz), antibodies to glucagon (1:100 Santa Cruz), and antibodies to nestin (1:200 courtesy of Dr. Conrad Messam, NINDS, NIH). Santa Cruz ABC staining system was used to visualize all antibody staining.

The cell cultures were comprised of multiple cell types as exemplified by differential staining patterns using cytokeratin 19, vimentin, and nestin. Nestin was expressed in all cultures to varying degrees. One culture, after, a period of conscientious neglect (see Cornelius et al., “In vitro-generation of islets,” 271-77; Ramiya et al., “Reversal of insulin-dependent diabetes,” 278-82), was comprised solely of nestin-positive cells. These cells were also vimentin-positive and cytokeratin-negative. No cells stained for insulin and glucagon. This culture was used in the following steps.

Culture on Poly-D-Lysine

After three months of culture in tissue culture plates, nestin-positive cells were grown at 37° C. and 5% CO₂ on tissue culture treated plates pre-coated with poly-D-lysine (5 micrograms per cm², BD Laboratories), using RPMI (Gibco) supplemented with 10% FBS, 100 mM penicillin, and 10 mM streptomycin.

Initially, most cells attached to the plates and began to spread onto the matrix within 15 minutes. However, instead of maintaining an even cell distribution on the plate as a monolayer, over two days, cells began to aggregate, forming a patchy network of epithelial cells. At two to three days, the culture began forming islet-like cell aggregates,

After two weeks, these aggregates were stained with dithizone (diphenylthiocarbazone, Sigma) to detect the presence of insulin granules. Dithizone stain was made by mixing 10 mg dithizone, 3 ml ethanol, and 3 drops of 30% NH₄OH. Five drops of the mixture were diluted in 2 ml phosphate buffered saline to stain cell aggregates for insulin. Stained cells became evident within 20 minutes. (Latif, Z. A., J. Noel, and R. Alejandro. A simple method of staining fresh and cultured islets. Transplantation 45, no. 4 (1988): 827:30.) Discreet areas within the cell aggregates were characteristically stained bright red. Further staining with insulin antibody showed more generalized staining of the cell aggregates.

Implantation and In Vivo Function Tests

Functionality was tested in vivo using a diabetic mouse model. Diabetes was induced into severe combined immunodeficient (“SCID”) mice by injection of streptozotocin (200 mg/kg), resulting in increased glucose concentrations from normal (80-110 mg/dl) to over 300 mg/dl within a few days after injection.

In preliminary experiments, when nestin-positive cell aggregates were implanted beneath the kidney capsules of diabetic mice, the blood glucose concentration dropped precipitously in many animals, leading to hypoglycemia and death without obvious signs of infection or other trauma. In the experiment described here, in order to decrease animal mortality and reduce potential glucose shock experienced by implanted cells, a slow release insulin pellet (Linshin, Inc., Canada) was placed subcutaneously to lower the blood glucose concentration.

Diabetic SCID mice received one of three procedures: (1) insulin pellets only; (2) nestin-positive cell aggregates and insulin pellets concurrently; and (3) insulin pellets followed by nestin-positive cell aggregates two days later. Five mice received the first procedure, insulin pellets only. Five mice received the second procedure, nestin-positive cell aggregates and insulin pellets concurrently. Six mice received the third procedure, insulin pellets followed by nestin-positive cell aggregates two days after placement of the insulin pellet. For the second and third procedures, about 10,000 nestin-positive cell aggregates were implanted. Blood glucose concentrations were measured about every four days, for a period of over 60 days.

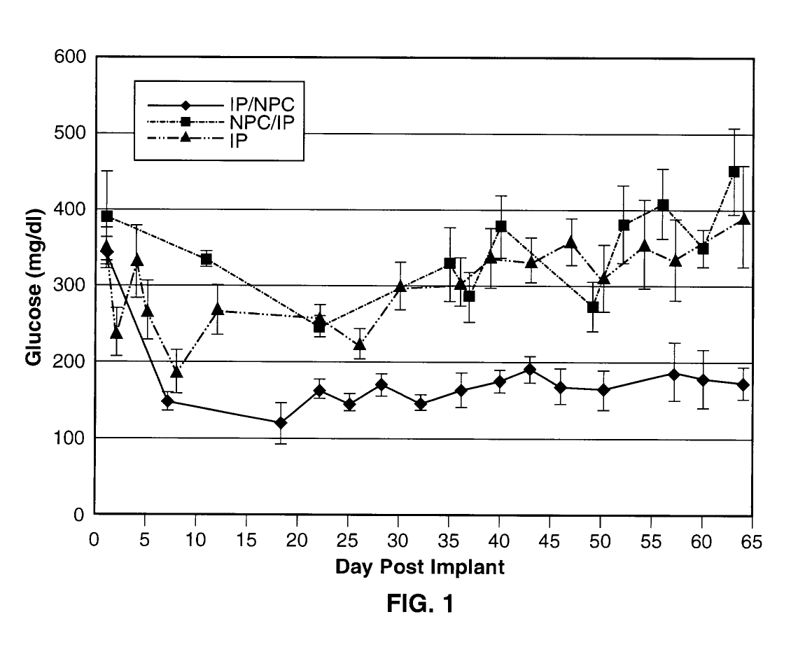

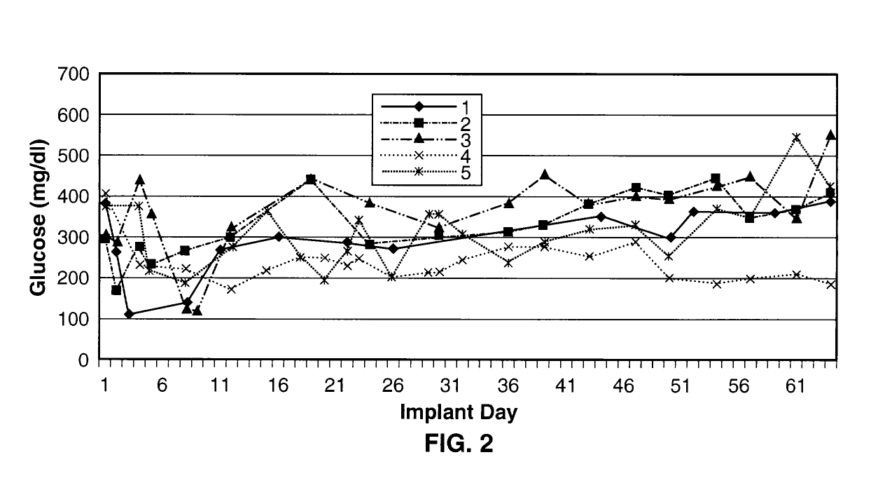

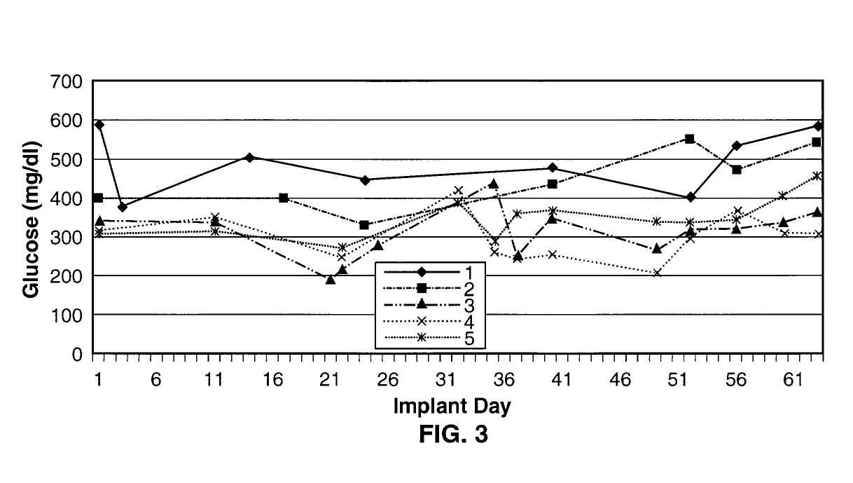

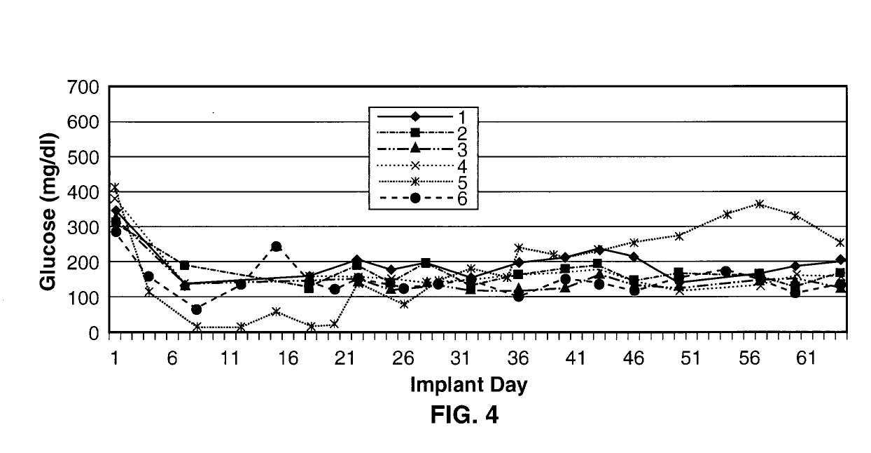

FIG. 1 shows the mean blood glucose concentrations of the mice treated with each of the three procedures. Mice that received the first procedure, insulin pellets only, are shown with the line indicated “IP.” Blood glucose concentrations in these mice dropped initially and then continued to increase over time to levels above 300 mg/dl. Mice that received the second procedure, nestin-positive cell aggregates and insulin pellets concurrently, are shown with the line indicated “NPC/IP.” Blood glucose concentrations in these mice dropped gradually, although never to normal levels, and then increased over time to levels above 300 mg/dl. Mice that received the third procedure, insulin pellets followed by nestin-positive cell aggregates two days later are shown with the line indicated “IP/NPC.” Blood glucose concentrations in these mice dropped quickly to levels close to normal (between about 120 mg/dl and about 180 mg/dl) and stayed at those levels for the entire period. FIGS. 2, 3, and 4, respectively, show the blood glucose concentrations of the individual mice described in FIG. 1. FIG. 2 shows those who received insulin pellets only; FIG. 3 shows those who received nestin-positive cell aggregates and insulin pellets concurrently; and FIG. 4 shows those who received insulin pellets followed by nestin-positive cell aggregates two days later.

Thus, only animals receiving the third procedure maintained glucose concentrations below 200 mg/dl for over 60 days. These results demonstrate that nestin-positive cell aggregates differentiate into cells capable of producing insulin and lowering blood glucose concentrations when implanted into diabetic mice.

The data further indicate that physiologic glucose concentration at the time of implantation of the nestin-positive cell aggregates affects the ability of nestin-positive cell aggregates to differentiate into functional islets. As stated above, nestin-positive cell aggregates implanted into overtly hyperglycemic mice were unable to improve blood glucose concentrations. Only mice implanted with nestin-positive cell aggregates after improvement of their glucose concentrations (i.e. those who received the third procedure, nestin-positive cell aggregates two days after receiving the insulin pellet) were able to maintain concentrations below 200 mg/dl for over 60 days (see FIG. 1, “IP CNPc”). The data also indicate that severe hypoglycemia compromises the viability of nestin-positive cell aggregates. FIG. 4 shows daily blood glucose concentrations for the six individual mice given the third procedure, insulin pellets followed by nestin-positive cell aggregates two days later, shown in FIG. 1 as “IP/NPC.” As shown in FIG. 4, in one of these mice, the blood glucose concentration fell to as low as 13 mg/dl and was below 30 mg/dl for at least five days. In this animal, when the effect of the insulin pellet wore off, by about day 35, the nestin-positive cell aggregates were unable to maintain normoglycemia, and the blood glucose concentration slowly increased to over 300 mg/dl.

The invention has been described above with reference to the preferred embodiment. Those skilled in the art may envision other embodiments and variations of the invention which fall within the scope of the claims.

I claim:

1. A method for isolating pancreatic cells capable of differentiating into beta cells, comprising:
isolating pancreatic cells;
culturing said pancreatic cells, wherein said culturing results in an increase in the number of cells that express nestin, as shown by nestin-positive staining;
culturing said nestin-positive cells on poly-D-lysine;
identifying an islet-like cell aggregate in said culture of nestin-positive cells; and,
isolating said islet-like cell aggregate, wherein said islet-like cell aggregate will function to lower the blood glucose concentration of a diabetic animal, after transplantation into said animal.

2. The method of claim 1, wherein said pancreatic cells are isolated from a human.

3. The method of claim 1, wherein, before culturing on poly-D-lysine, said nestin-positive cells are also vimentin-positive.

4. The method of claim 1, wherein, before culturing on poly-D-lysine, said nestin-positive cells are also cytokeratin-negative.

5. The method of claim 1, wherein, before culturing on poly-D-lysine, said nestin-positive cells are also insulin-negative and glucagon-negative.

6. The method of claim 1, wherein said culturing on poly-D-lysine is done for a period of about one to two days.

7. The method of claim 1, wherein, after culturing on poly-D-lysine, said islet-like cell aggregate expresses insulin.

8. The method of claim 1, wherein the diabetic animal into which the islet-like cell aggregate is transplanted is a human.

9. The method of claim 1, wherein the diabetic animal into which the islet-like cell aggregate is transplanted is an animal whose blood glucose concentration is well-controlled.

10. A method for differentiating islet precursor cells into beta cells, comprising:
isolating pancreatic cells;
culturing said pancreatic cells to enrich the culture for nestin-positive cells;
culturing said pancreatic cells on poly-D-lysine;
identifying an islet-like cell aggregate in said culture of pancreatic cells;
isolating said islet-like cell aggregate, wherein said islet-like cell aggregate expresses insulin.

11. The method of claim 10, wherein said pancreatic cells are isolated from a human.

12. The method of claim 10, wherein said enriching of the culture for nestin-positive cells is accomplished by conscientious neglect.

13. The method of claim 10, wherein, before culturing on poly-D-lysine, said nestin-positive cells are also vimentin-positive.

14. The method of claim 10, wherein, before culturing on poly-D-lysine, said nestin-positive cells are also insulin-negative and glucagon-negative.

15. The method of claim 10, wherein a cell of said islet-like cell aggregate will function as a beta cell after transplantation into a diabetic animal.

16. The method of claim 15, wherein the diabetic animal into which the islet-like cell aggregate is transplanted is a human.

17. The method of claim 15, wherein the diabetic animal into which the islet-like cell aggregate is transplanted is an animal whose blood glucose concentration is well-controlled.
